(19) United States
(12) Patent Application Publication
(10) Pub. No.: US 2005/0009759 A1
(43) Pub. Date: Jan. 13, 2005

(54) Derivatives of etoposide and analogs, and pharmaceutical compositions containing them

(76) Inventors: Claude Monneret, Paris (FR); Frederic Schmidt, Vincennes (FR)

Correspondence Address: YOUNG & THOMPSON, 745 SOUTH 23RD STREET, 2ND FLOOR, ARLINGTON, VA, 22202, US

(21) Appl. No.: 10/493,597

(22) PCT Filed: Oct. 25, 2002
(86) PCT No.: PCT/EP02/11965

(30) Foreign Application Priority Data
Oct. 26, 2001 (EP) 01402787.4

(51) Int. Cl. A61K 31/7048

(52) U.S. Cl. 514/27; 536/53

(57) ABSTRACT

The invention relates to compounds having the following formula (I), in which: Ra represents a sugar moiety, an arylamino group, or an alkyl group comprising at least one amino group, Rb represents an halogen atom, an halogenoalkyl group, a nitro group, or a group —NR(COR′) in which R and R′, independently from each other, represent an alkyl group, R1 represents H, or a protecting group for COOH group, R2, R3, and R4, independently from each other, represent H, or a protecting group for OH group. The invention also relates to the use of such compounds in pharmaceutical compositions for the treatment of cancers.

The invention relates to new derivatives of etoposide, and derivatives of compounds derived from etoposide, and to their use in pharmaceutical compositions for the treatment of cancers.

Human β-glucuronidase is an essential catabolic enzyme, an exoglycosidase cleaving glucuronosyl O-bonds present in lysosomial glycosylaminoglycans such as chondroitin sulfate and hyaluronic acid.

In addition, human β-glucuronidase plays a role in the deconjugation of some endogenous substances. The activity of this enzyme in the plasma and extracellular compartment is very low, since the enzyme is almost completely localized into the lysosomes. However, elevated activity of β-glucuronidase in tumor tissues has been observed for a long time and reported by different authors [Fishman W H and Anlyan A J [J. Biol. Chem. 169, 449 (1947)], Anghileri L J & Miller E S, Oncology 25, 1932 (1971); Fishman W H et al. Cancer 12, 240 (1959); Young C W et al. Cancer 38, 1887 (1976). Warenius H M & al. Br. J. Cancer 45, 27 (1982); Boyer & Tannock Adv. Cancer Res. 60, 269 (1993); Ruben D. U.S. Pat. No. 5,340,803].

Similarly, in some inflammatory diseases such as rheumatic arthritis, an increase in the enzyme level has been noted. [Caygil J C& Pitkeathy D A Ann.Rheum. Dis. 25, 137 (1966); Weissman et al. J. Exp. Med. 134, 521 (1971)].

Nevertheless, selective induction of the activation of glucuronide prodrugs into drugs taking advantage of the increased concentration of β-glucuronidase in the tumor has not been widely used. Only few examples with aniline mustards have been reported. [Connors T A & Whisson M E, Nature 210, 866 (1966); Connors T A et al. Biochem. Pharmacol. 22, 1971 (1973); Double J A, Workman P A, Cancer Treat. Rep. 61, 909 (1977)].

In 1995, reinvestigation of the β-glucuronidase level in several tumor tissues has been undertaken by Bosslet et al. [Tumor Targeting 1, 45 (1995)]. It was unambiguously shown, by enzyme histochemistry, that necrotic areas in human cancers are the sites in which lysosomal β-glucuronidase is liberated extracellularly in high local concentration. This study, carried out by immunochemistry, also demonstrated that the cells responsible for the liberation of the enzyme are mainly acute and chronic inflammatory cells. Later on, the same authors elucidated the mechanism enabling tumor selective prodrug monotherapy [Bosslet et al. Cancer Res. 58, 1195 (1998)]. According to IHC investigations, extracellular β-glucuronidase originates from monocytes and granulocytes concentrated within the necrotic areas but not (or only to a low extent) from tumor cells. Furthermore, the enzyme activity detectable by study enzyme histochemistry in necrosis of xenografts in mice, did not stain with monoclonal antibody, selective for human β-glucuronidase, and therefore is not from human origin.

Taking these data into consideration, relevant results have been observed in a broad panel of tumors (human tumor xenografts) with a glucuronide prodrug of doxorubicin, HMR 1826 [Florent J C et al. J. Med. Chem. 41, 3572 (1998)]. Prodrug monotherapy in such human tumors generates superior therapeutic effects versus standard chemotherapy. An enhanced uptake of doxorubicin in bronchial carcinoma was subsequently observed on isolated and perfused human lung model. The level of doxorubicin after lung perfusion with HMR 1826 was about 7-fold higher than after perfusion with doxorubicin itself [Mürdter T E et al. Cancer Res. 57, 2440 (1997)].

Next experiment also showed increasing level of β-glucuronidase activity in pancreatic cancer. This may represent a potential role in drug targeting, especially in the treatment of pancreatic carcinoma by using glucuronide prodrugs of anticancer agents.

Evidence based on all these examples indicates that this approach using a glucuronide prodrug may be useful in increasing the delivery of oncostatic drugs in tumors in human (de Groot et al. Current Medicin. Chem. 8, 1093 (2001).

On the basis of these observations, a glucuronide prodrug synthesis program was initiated and podophyllotoxin prodrugs were included among the cytotoxic compounds investigated in this program.

Etoposide, or VP-16, is a semi-synthetic compound which exerts its antitumor activity by stabilization of the ternary complex involving the drug, DNA and Topoisomerase II. Established indications of etoposide are testicular and small-cell lung cancers; the use in pediatrics for the treatment of neuroblastoma is also well known. Etoposide is also indicated in cancer leukemia, and Kaposi's sarcoma.

In spite of this widespread clinical use, there is a limitation due to its very poor water-solubility. Formulation with Tween 80, polyethylene glycol and ethanol results in acute mortality. To solve this problem in the 1990's, the group of Bristol-Myers Squibb initiated a program to discover an appropriate prodrug. This led to the development of etoposide phosphate, BMY-404811 [Saulnier et al. Bioorg. Med. Chem. Lett. 1994, 4, 2567]. Etoposide phosphate (Etopophos) is rapidly converted to the parent drug in vivo and, therefore, has been introduced in clinics with the same profile as etoposide itself. On this account, although esterification of the phenol function significantly decreases, not only the activity, but also the toxicity, both are almost restored through the enzymatic cleavage. This indicates that there was no marked gain in selectivity with this type of prodrug.

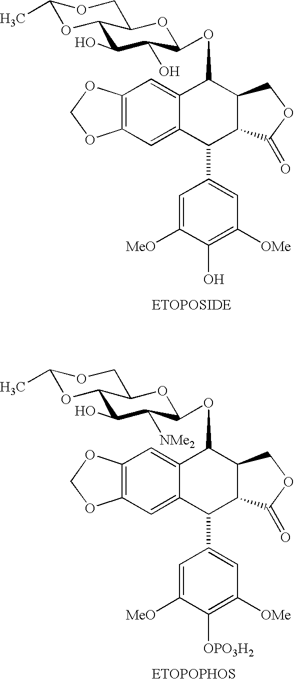

Simultaneously to this discovery, Bristol-Myers group developed an amino-derivative, NK 611. This compound [Rassmann et al. Invest. New Drugs 1996, 14, 379-386] is currently undergoing phase I evaluation and further introduction in phase II is expected.

Other modifications introduced at C-4 have led other groups to find compounds in which the sugar moiety has been replaced by an arylamino group such as a 4-fluoroaniline (in NPF) (Lee et al., J. Med. Chem., 1990, 33, 364) or by a dimethylethylamino side-chain (TOP 53)(Utsugi et al., Cancer Res., 1996, 56, 2809). Both derivatives are presently under clinical trials.

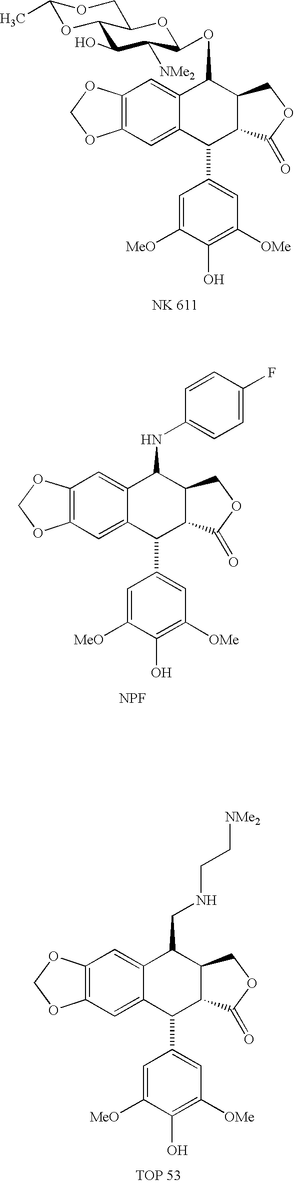

In order to obtain an adequate enzymatic hydrolysis turn-over, three-comparment prodrugs have been designed by the Inventors according to the proposal of Katzenellenbogen [Carl P L et al. J. Med. Chem. 24, 479 (1981)], concept which previously developed by the Inventors with glucuronide prodrugs of anthracyclines [Andrianomenjanahary S et al. Bioorg. Med. Chem. Lett. 2, 1093 (1992); Gesson J P et al. Anti-Cancer Drug Design 9, 409 (1994); Azoulay M. et al. Ibid 10,-441 (1995)]; Schmidt F. et al. Bioorg. Med. Chem. Lett. 7, 1071 (1997); Florent J C et al. J. Med. Chem. 41, 3572 (1998); Desbene S. et al. Anti-Cancer Drug Design 13, 955 (1998)], of phenolic nitrogen mustards (Lougerstay-Madec R. et al. Ibid 13, 995 (1998)], of M.D.R. modulators [Desbene et al. Ibid 14, 93 (1999)], of 5-fluorouracil [Lougerstay-Madec R. J. Chem. Soc. Perkin Trans I, 1369 (1999)] and more recently, of taxol. [ Schmidt F. et al Eur. J. Org. Chem. 2129 (2001)].

The self-immolative spacer described in the present invention is the same that those already reported for preparing nitrogen mustard prodrugs [Schmidt F. et al. Bioorg. Med. Chem. Lett. 7, 1071 (1997)].

The Inventors give for the first time evidence that etoposide and derivatives compounds such as NK 611, NPF, and TOP 53 mentioned above, can be linked to a glucuronide moiety via said spacer, without encountering particular problems related to spatial organization of the final prodrug.

The use of this spacer is advantageous because it allows an easy access of β-glucuronidase to the glucuronide moiety. The glucuronide-spacer-etoposide is thus a far better substrate for β-glucuronidase as compared to spacer-less compounds such as glycosyl-etoposide (EP 0 423 747 A) in which the glycosyl moiety lacks accessibility. The prodrug activity of such spacer-less compounds is therefore severely impaired because the rate of etoposide release following hydrolysis is too low.

Furthermore, the Inventors give the demonstration that, as soon as the enzymatic hydrolysis has occurred, self-immolative decomposition of the spacer is observed, as depicted below with liberation of etoposide and of the cyclized spacer.

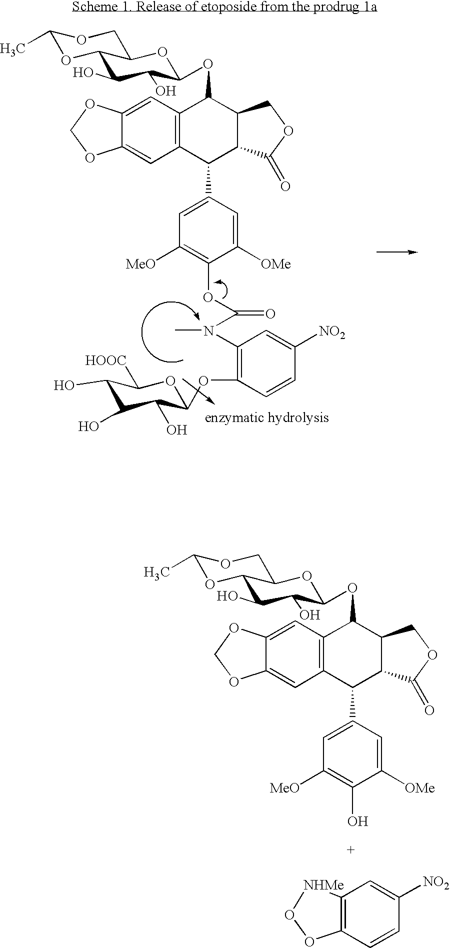

The aim of the present invention is to provide new prodrugs of etoposide, and of derivatives such as NK 611, NPF, TOP 53 and other 4-substituted 4-epi-4′-demethoxypodophyllotoxin derivatives endowed with antitumor activity, their method of preparation and their use.

More particularly, the aim of the present invention is to provide water-soluble prodrugs of etoposide and derivatives. These prodrugs, which are stable in plasma, selectively deliver etoposide or derivatives in necrotic areas of tumors due to the increased level of the β-glucuronidase enzyme.

Advantageously, prodrugs of the invention have selective activity within the tumors, while side-effects in normal tissues are minimized.

The present invention relates to compounds having the following formula (I): 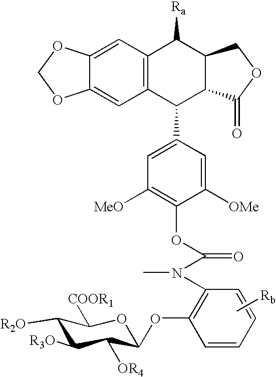
in which:
Rₐ, represents a sugar moiety, an arylamino group, or an alkyl group, advantageously from 1 to 10 carbon atoms, said alkyl group comprising at least one amino group,
R_{b} represents an halogen atom, an halogenoalkyl group, advantageously from 1 to 5 carbon atoms, a nitro group, or a group —NR(COR′) in which R and R′, independently from each other, represent an alkyl group, advantageously from 1 to 5 carbon atoms,
R₁ represents H, or a protecting group for COOH group,
R₂, R₃, and R_{4,} independently from each other, represent H, or a protecting group for OH group.

The invention relates more particularly to compounds described above of formula (I) wherein R₁, R₂, R₃, and R₄ represent H.

The invention more particularly concerns compounds as described above, of formula (I) wherein Rₐ represents:
a sugar moiety, selected among the derivatives of glucose, such as the glucose methylacetal of the following formula: 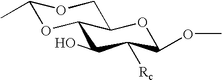
   wherein R_{c} represents an hydroxyl or an amino group such as —N(CH₃)₂,
or an arylamino group, and more particularly a group of the following formula:
   —HN—C₆H₄R_{d}
   wherein R_{d} represents an halogen atom, or a nitro group, such as the arylamino group selected among 4-nitroaniline, or 4-fluoroaniline,
or an alkyl group from 5 to 10 carbon atoms comprising at least one amino group, and more particularly a linear alkyl chain comprising two nitrogen atoms in the chain, such as the [(dimethylamino)ethyl]N-methylamino)ethyl group.

The invention relates more particularly to compounds as described above, of formula (I) wherein R_{b} represents NO₂, F, Cl, CF₃, or a group —NR(COR′) in which R and R′, independently from each other, represent an alkyl group from 1 to 5 carbon atoms.

Preferred compounds according to the invention have the following formulae: 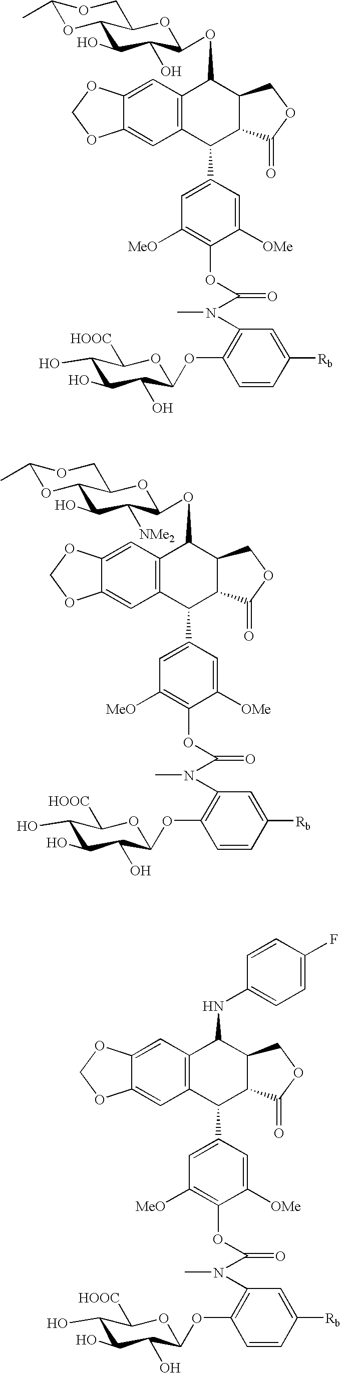 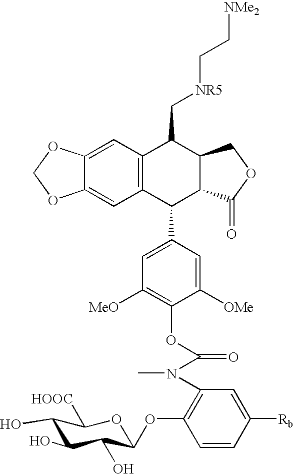
wherein R_{b} represents NO₂, F, Cl, CF₃, or a group —NR(COR¹) in which R and R′, independently from each other, represent an alkyl group from 1 to 5 carbon atoms, and R₅ represents H or CH₃.

A more particularly preferred compound according to the invention, has the following formula:

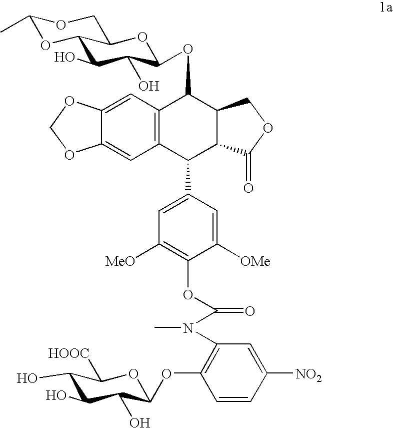

As mentioned above, compounds of the present invention present the specificity to act as prodrugs capable of releasing in the organism the drugs of formula 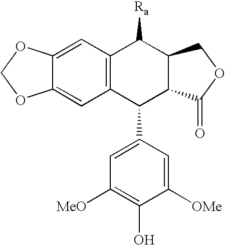
in which Rₐ is defined above.

Prodrugs according to the present invention have the following characteristics:
they are highly detoxified,
the in vitro determination of enzymatic hydrolysis kinetics with β-glucuronidase from E. coli, shows that said hydrolysis is carried out within a laps of time compatible with a therapeutic use of said prodrugs (approximately 50% of release of prodrug in 25 min),
the prodrugs are stable (more than 90% of the prodrug remaining after 24 hours at 37° C. in a phosphate buffer),
the prodrugs are soluble in aqueous solvents, their solubility being approximately of 20 mg/ml (i.e. much more soluble than the etoposide, the solubility of which being of 0,1 mg/ml).

The invention also concerns pharmaceutical compositions comprising at least one compound of formula (I) as defined above, and more particularly at least one compound of formula (I) wherein R₁, R₂, R₃, and R₄ represent H, or a salt thereof, in association with a suitable pharmaceutical carrier.

The invention relates more particularly to pharmaceutical compositions as defined above, comprising at least one of the following compounds: 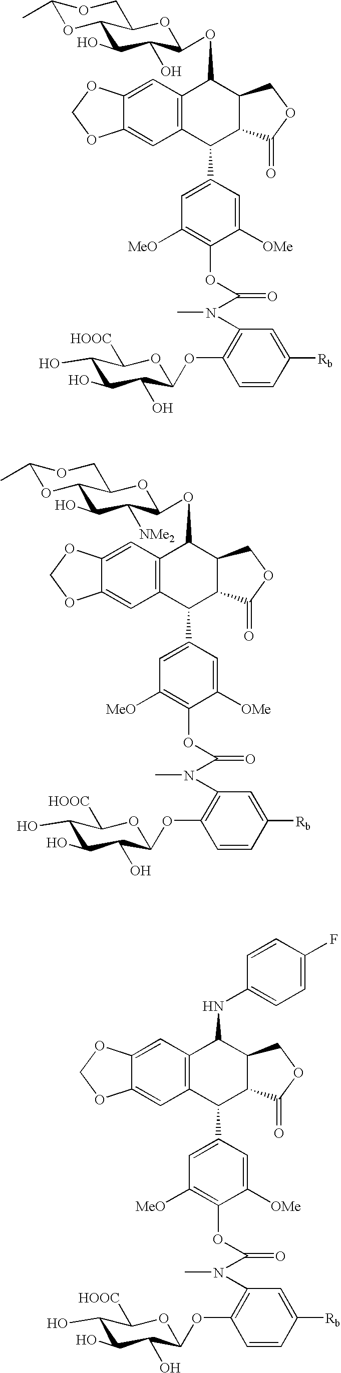 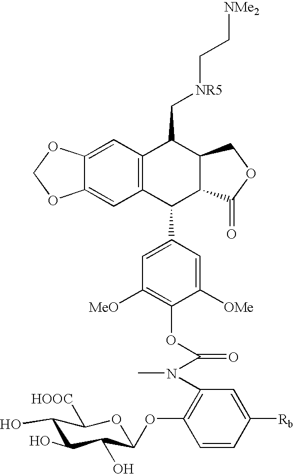
wherein R_{b} represents NO₂, F, Cl, CF₃, or a group —NR(COR′) in which R and R′, independently from each other, represent an alkyl group from 1 to 5 carbon atoms, and R₅ represents H or CH₃.

Preferred pharmaceutical compositions according to the invention, are those comprising at least the following compound:

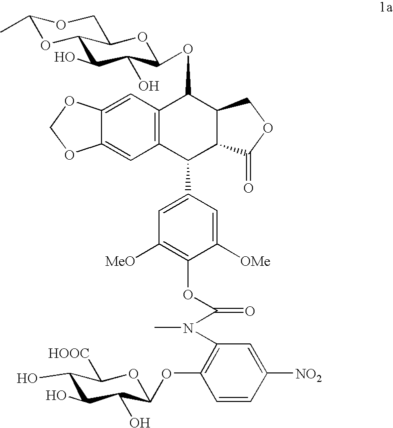

Advantageously, pharmaceutical compositions according to the invention, are in a suitable form for oral administration, or for administration by injection, such as intravenous route.

Preferred pharmaceutical compositions according to the invention, are characterized in that the dosage of the compounds of formula (I) is comprised between approximately 100 mg/m²/day, and approximately 200 mg/m²/day (when based on etoposide equivalent), during approximately 5 days.

The invention also relates to the use of a compound of formula (I) as defined above, and more particularly to the use of at least one compound of formula (I) wherein R₁, R₂, R₃, and R₄ represent H, or a salt thereof, for the manufacture of a drug for the treatment of cancers such as lung cancer, testicular cancer, Kaposi's sarcoma, lymphoma, and leukemia.

The invention also concerns a process for the preparation of a compound as defined above of formula (I), characterized in that it comprises the following steps:
amine activation of the following compound of formula A 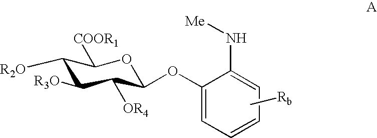
   wherein:
R₁ represents a protecting group for COOH group, such as a benzyl or a methyl group,
R₂, R₃, and R₄ represent a protecting group for OH group, such as a terbutyldimethylsilyl or acetate group,
R_{b} is such as defined above,
   by treatment of said compound A with phosgene in order to obtain the following compound of formula B: 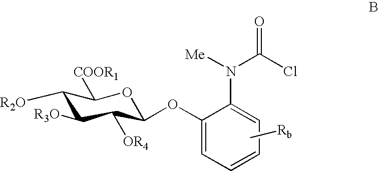
   wherein R₁, R₂, R₃, R₄, and R_{b} are such as defined above,
coupling of compound B obtained above, with the following compound of formula C, 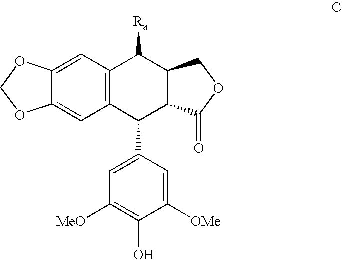
   wherein Rₐ is such as defined above, in order to obtain the following compound D: 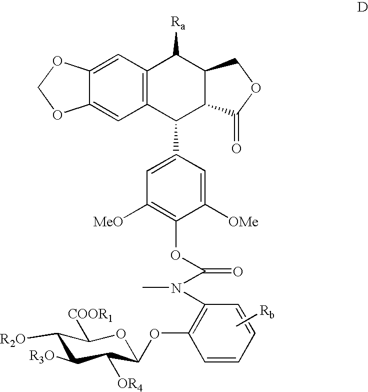
   wherein:
R₁ represents a protecting group for COOH group, such as a benzyl or methyl group,
R₂, R₃, and R₄ represent a protecting group for OH group, such as a terbutyldimethylsilyl or acetate group,
Rₐ and R_{b} are such as defined above,
deprotection of the OH groups of compound D, for instance with HF/pyridine when R₂, R₃, and R₄ represent a terbutyldimethylsilyl group, in order to obtain the following compound E: 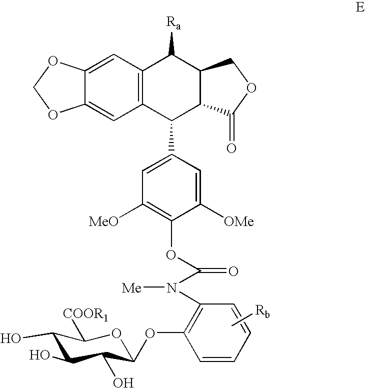
   wherein:
R₁ represents a protecting group for COOH group, such as a benzyl or methyl group,
Rₐ and R_{b} are such as defined above,
deprotection of the COOH group of compound E, for instance with cyclohexadiene over palladium when R₁ represents a benzyl group, in order to obtain the following compound F: 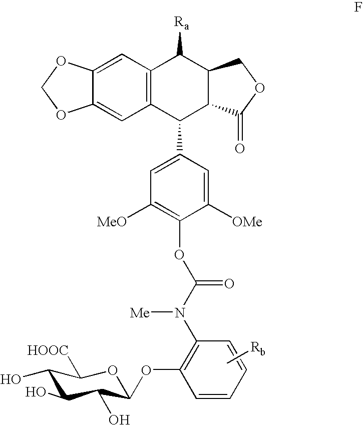
   wherein Rₐ and R_{b} are such as defined above.

The invention also relates to compounds defined above of formula (I), used as intermediary products in the process mentioned above, said compounds corresponding to those of formula (I) wherein:
R₁ represents a protecting group for COOH group, such as a benzyl or methyl group,
and/or, R₂, R₃, and R₄ represent a protecting group for OH group, such as a terbutyldimethylsilyl or acetate group.

The invention relates more particularly to compounds used as intermediary products defined above, having the following formulae: 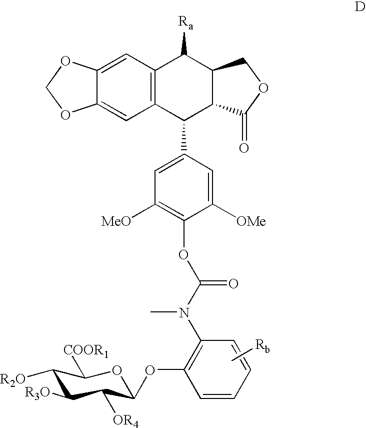
wherein
R₁ represents a protecting group for COOH group, such as a benzyl or methyl group,
and R₂, R₃, and R₄ represent a protecting group for OH group, such as a terbutyldimethylsilyl or acetate group,
Rₐ and R_{b} are such as defined above, 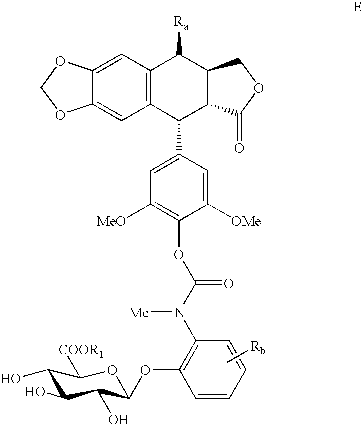
   wherein R₁, represents a protecting group for COOH group, such as a benzyl or methyl group, and Rₐ, and R_{b} are such as defined above.

The invention will be further described in the detailed description of the synthesis of compounds of formula (I), and of the study of their properties.

Synthesis of prodrugs according to the invention required the use of protecting groups compatible with the sensitivity of etoposide structures, or derivatives thereof, under basic conditions. It is well known that, even under slightly basic conditions, the trans-fused lactone as present in podophyllotoxin derivatives easily epimerise to give cis-fused picropodophyllin analogs, which are devoid of antitumor activity (Gensler, W.; Gatsonis, C. J. Chem. Soc., 1966, 31, 3224-3227, Aso, Y.; Hayashi, Y.; Yoshioka, S.; Takeda, Y.; Kita, Y.; Nishimura, Arata, Y. Chem. Pharm. Bull., 1989, 37, 422-424).

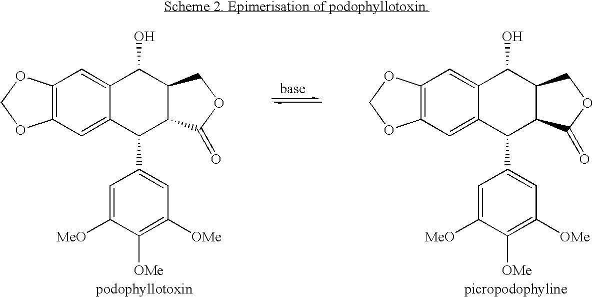

Therefore in order to avoid this problem, the synthesis of the prodrug 1a was achieved as follows.

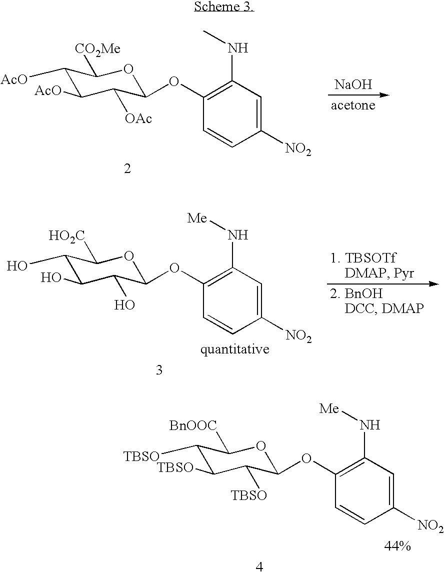

The hydroxyl and carboxyl protecting groups as present in intermediate 2 (Desbene, S.; Dufat-Trinh van, H.; Michel, S.; Tillequin, F.; Koch, M.; Schmidt, F.; Florent, J.-C.; Monneret, C.; Straub, R.; Czech, J.; Gerken,. M.; Bosslet, K. Anti-cancer Drug Design, 1999, 14, 93-106) were removed and then reprotected as TBDMS ethers for the hydroxyl groups and as a benzyl ester for the carboxylic acid, to successively afford 3 and 4.

Next steps involved amine activation of 4 by treatment with phosgene. Subsequent coupling of 5 with etoposide under controlled conditions (etoposide, 1 equiv.; carbamoyl chloride, 1.25 equiv.; and DMAP, >2 equiv.) led to the protected prodrug 6.

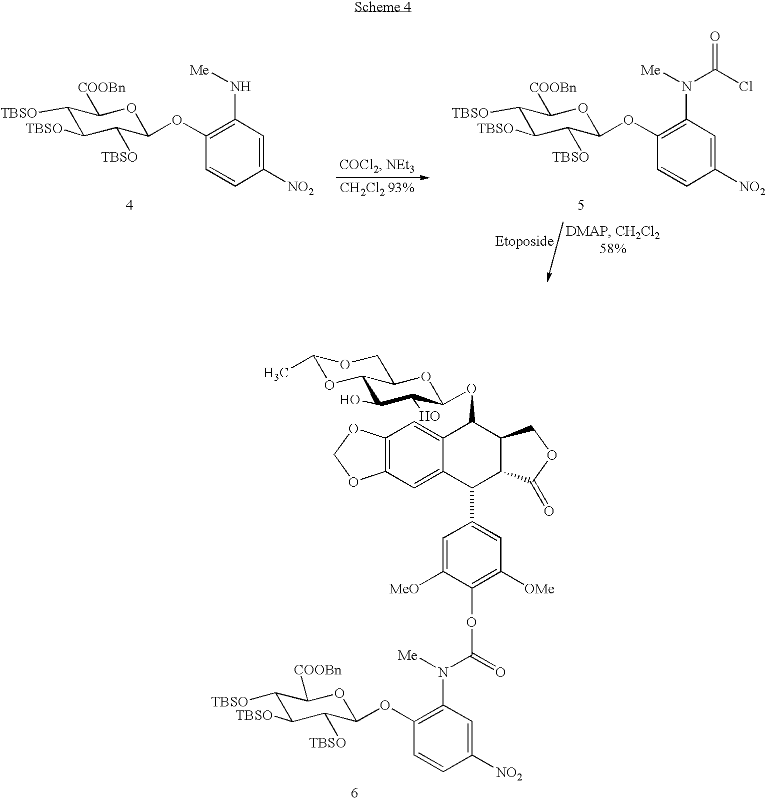

Deprotection of the glucuronide moiety was then achieved to convert 6 into prodrug 1a. The TBDMS groups were removed with HF/Pyridine giving 7 and the benzyl ester with cyclohexadiene over palladium (Jeffrey, P. Mac Combie. S. J. Org. Chem., 1982, 47, 587-590, Desiel, R. Tetrahedron Lett., 1987, 28, 4371-4372). The overall yield starting from etoposide is 15%.

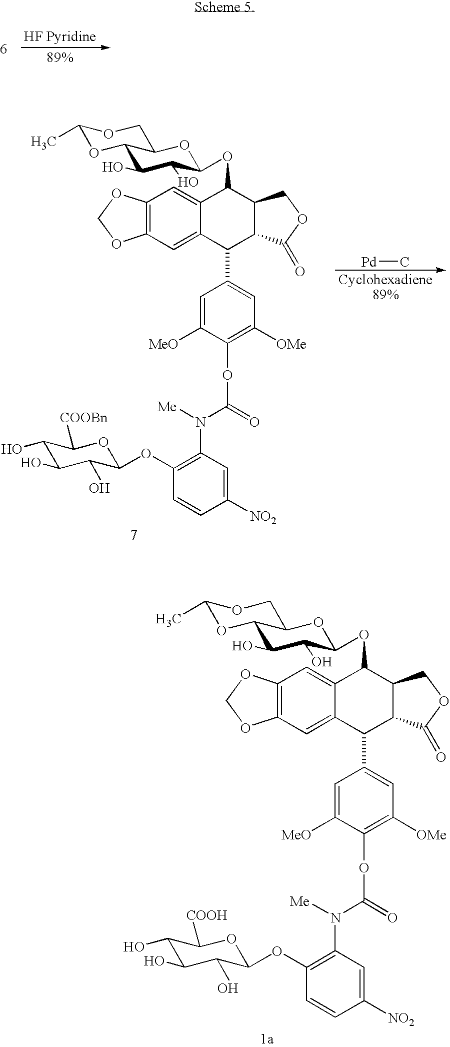

Biological Activity

Solubility

In water and under comparable conditions, prodrug 1a is approximately 200-fold more soluble than the corresponding drug. Thus, while the solubility of etoposide is about 0.1 mg/mL, the solubility of 1a is about 20 mg/mL.

Cytotoxicity

On L1210 cell line, prodrug 1a gave an IC₅₀ value of 50.2 μM. After hydrolysis by β-glucuronidase, increased cytotoxicity was obtained with an IC₅₀ value of 0.93 μM were closely related to that of etoposide itself (0.834 μM). This means that the prodrug was detoxified by a factor of about 50.

Stability

The stability of 1a was followed by HPLC measurements in buffer solution at pH 7 for 24 hours. More than 90% of the prodrug was recovered after that time, meaning that the prodrug 1a is stable in vitro.

Kinetics of Drug Release

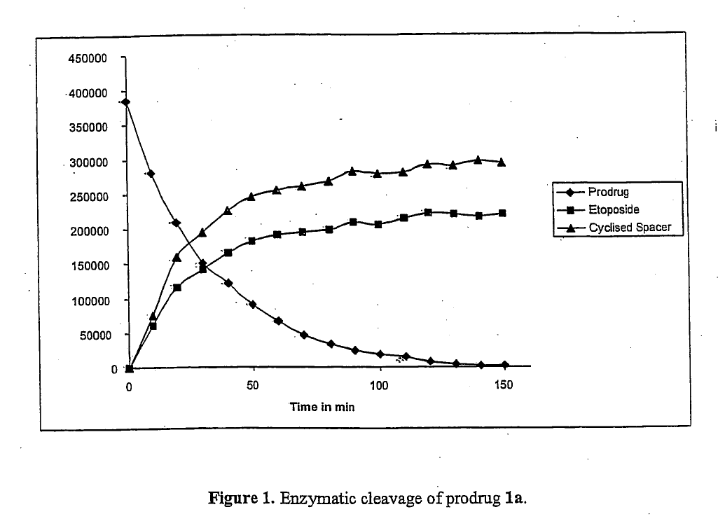

Prodrug 1a (500 μg/mp) was incubated with E. coli β-D-glucuronidase (20 μ/mL). Aliquot samples were analysed by IPLC at different times (FIG. 1: Enzymatic cleavage of prodrug 1a).

Examination of the curve indicates that the prodrug 1a is rapidly hydrolysed, the only products detected being the etoposide 1 and the cyclised spacer. No intermediate containing the spacer still attached to the etoposide was seen. This is consistent with a fast enzymatic cleavage (half-live of 1a is <25 min) and a fast cleavage of the spacer.

It is also assumed that the liberated compound is indeed etoposide and not picroetoposide which was synthesised following a described procedure (Meresse, P.; Bertounesque, E.; Imbert, T.; Monneret, C. Tetrahedron, 1999, 55, 12805-12818). HPLC examination by comparison of the retention times was in agreement with the fact that, during all the synthesis, the trans-fused lactone was not epimerised into cis-fused lactone.

Experimental

Melting points (mp) were taken on a Koffler Bench and are uncorrected. Optical rotations were obtained on a Perkin-Elmer 241 polarimeter (589 nm). Specific rotations ([α]_{D}) are reported in deg/dm, and the concentration (c) is given in g/100 mL in the specific solvent. Infared spectra were recorded on a Perkin-Ehmer 1600 FTIR spectrometer (v in cm⁻¹). ¹H-NMR (300 MHz) and ¹³C-NMR (75 MHz) spectra were recorded on Bruker AC 300 spectrometer —chemical shifts δ in ppm and J in Hz. Chemical ionization (CI—MS; NH₃, positive ion mode) or FAB (positive ion mode) mass spectra, were recorded on a Nermag R 10-10C spectrometer. Electrospray ionisation mass spectra (ESI-MS) were acquired using a quadripole instrument with a mass of charge (m/z) range of 2000. The Nermag R 10-10 mass spectrometer used was equipped with an analytical atmospheric pressure electrospray source. The chromatography were conducted over silica gel (Merck 60 (230-400 Mesh).

For the NMR description, the following numerotation was chosen: “a” for aromatic, “e” for etoposide, “g” for glucose, “G” for glucuronic acid)

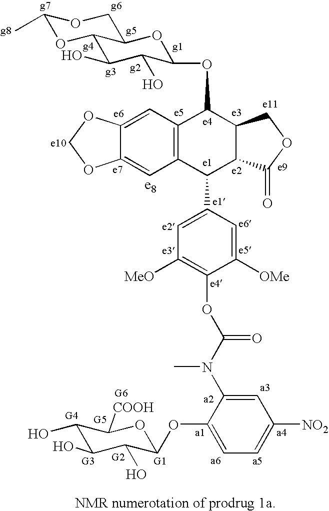

### 2-Methylamino-4nitrophenyl-β-D-glucopyranosiduronic acid 3

To a solution of 2 (2 g, 4.13 mmol) in 50 mL acetone at 0° C., a 1N NaOH aqueous solution (50 mL) was added dropwise. After 5 min of stirring at 0° C., the mixture was neutralized with 1N HCl at pH4, evaporated and purified by column chromatography (CH₃CN/H₂O: 80/20). The solid was heated in boiling methanol and filtrated to eliminate silica. After evaporation, 3 was obtained as a bright orange solid (100%). C₁₃H₁₆N₂O₉; mp 172° C.; [α]_{D}−53 (c 0.96 in MeOH); νₘₐₓ/cm^{-1 (KBr)} 3400 (O—H), 1588 (aromatics), 1530, 1343 (NO₂); δ_{H} (DMSO) 7.46 (1H, dd, Jm, 3, J₀ 9, a5), 7.19 (1H, d, Jₘ 3, a6), 7.12 (1H, d, Jₒ, 9, a3), 6.04 (1H, q, J 5, N—H), 5.68 (1H, br, s, OH), 5.13 (1H, br, s, OH), 4.85 (1H, d, J 7, G1), 3,57-3.17 (4H, G2, G3, G4, G5), 2.8 (3H, d, J 5, N—CH₃); δ_{C}(DMSO) 172.4(G6), 149.8 (a1), 143.0 (a2), 140.5 (a4), 113.3 (a5), 111.6 (a6), 102.3 (a3), 101.4 (G1), 75.6, 74.1, 73.0, 72.1 (G2, G3, G4, G5), 29.4 (NMe); m/z (ES⁻) 343 [M−H]⁻.

### Benzyl [2-methylamino-4-nitrophenyl-2,3,4-tri-O-(tert-butyldimethylsilyl)-β-D-glucopyranosid]uronate (4)

DMAP (0.1 g) was added to a solution of 3 (1.87 g, 5.43 mmol) in 20 mL of pyridine. The mixture was cooled to 0° C. and TBS trifate (12 mL, 52.3 mmol) was added dropwise. After 48 h at room temperature, the mixture was evaporated and the residue was taken in toluene (200 mL). The insoluble pyridinium triflate was filtered and the filtrate evaporated. The product, obtained as a yellow resin (3.64 g, 5.31 mmol) was used without any purification in the next step. A solution of DMAP (0.3 g, 2.45 mmol) in 20 mL CH₂Cl₂ was then added. After cooling to 0° C., benzyl alcohol (0.5 mL, 4.9 mmol) and DCC (1.095 g, 5.31 mmol) were successively added. After 12 hours at room temperature, the mixture was evaporated and poured into cyclohexane (250 mL). The insoluble urea was filtered. The filtrate was evaporated and purified by two successive chromatographies, the first with CH₂Cl₂ and the second with CH₂Cl₂/cyclohexane: 5/1. Compound 4 was isolated as a yellow resin (1.83 g, 44% from 3). C₃₈H₆₄NO₉Si₃; [α]_{D} −2.3 (c 0.98 in CHCl₃); νₘₐₓ/cm⁻¹(CDCl₃) 1762 (C═O ester), 1623 (aromatics), 1530, 1343 (NO₂); δ_{H}(CDCl₃) 7.53 (1H, dd, J₀ 9, Jm 3, a5), 7.35 (1H, d, Jm 3, a3), 7.33-7.26 (5 H, Ph), 6.83 (1H, d, J₀ 9, a6), 5.62 (1H, d, J 6 G1), 5.11 (s, 2 H, CH₂Ph), 4.59 (1H, q, J 5, NH), 4.52 (1 H, G3), 4.38 (1 H, G4), 4.02 (1H, t, J 6, G2), 3.87 (1H, d, J 3.5, G5), 2.86 (3H, d, J 5, N—CH₃), 0.91 (18 H, Si—C—CH₃), 0.86 (9 H, Si—C—CH₃), 0.15 (3H, Si—CH₃), 0.14 (3H, Si—CH₃), 0.12 (6H, Si—CH₃), 0.08 (3H, Si—CH₃), −0.01 (3H, Si—CH₃); δ_{C}(CDCl₃) 168.4 (G6), 148.8 (a1), 143.7 (a2), 140.5 (a4), 135.1 (Ph quaternary), 128.5-128.4-128.3 (Ph tertiary), 112.5 (a5), 112.0 (a6), 104.1 (a3), 98.9 (G1), 78.9 (G3), 77.2 (G5), 75.7(G2), 72.1 (G4), 67.0 (CH₂Ph), 29.4 (NMe), 25.7 (Si—C—CH₃), 18.0-17.9 (Si—C—CH₃) −4.5 −4.6 −4.7 −5 (Si—CH₃); m/z (CI) 777 [M+H]⁺.

### Benzyl [2-(N-chloroformyl-N-methylamino)-4-nitrophenyl-2,3,4-tri-O-(tert-butyldimethylsilyl)-β-D-glucopyranosid]uronate 5

To a solution of 4 (350 mg, 0.45 mmol) in 20 mL CH₂Cl₂ at 0° C., a solution of phosgene (700 μl 1.35 mmol) in toluene was added. Then triethylamine (1.13 mL, 8.16 mmol) was added dropwise. After 30 min at 0° C., the reaction was quenched with 10 mL water. The organic phase was separated and washed with 10 mL of brine, dried over magnesium sulfate and evaporated. The residue was purified by chromatography (EtOAc/cyclohexane: 1/13) to obtain 5 as a colourless viscous oil (352 mg, 93%). C₃₉H₆₃N₂O₁₀ClSi₃; [α]_{D} −5 (c 1 in CHCl₃); νₘₐₓ/cm⁻¹(CDCl₃), 1734 (C═O carbamoyl chloride), 1594 (aromatics), 1528, 1349 (NO₂); δ_{H}(CDCl₃) 8.24 (1H, dd, J₀ 9, Jₘ 3, a5), 8.14 (1H, d, Jₘ 3, a3), 7.34-7.28 (5H, Ph), 7.22 (1H, d, J₀ 9, a6), 5.73 (1H, d, J 5.5, G1), 5.07-5.05 (2H, 2 s, CH₂Ph), 4.43 (1 H, G3), 4.40 (1 H, G4), 3.96 (1H, G2), 3.88 (1H, d, J 3, G5), 3.25 (3H, s, N—CH₃), 0.95-0.86 (27 H, Si—C—CH₃), 0.08 (12 H, Si—CH₃), 0.07 (3 H, Si—CH₃), 0.02 (3 H, Si—CH₃); δ_{C}(CDCl₃) 168.0 (G6), 157.5 (NCOCl), 148.6 (a1), 142.1 (a2), 134.7 (a4), 133.1 (Ph quaternary), 128.3-128.1-125.7 (Ph tertiary), 125.5 (a5), 115.9 (a6), 99.3 (a3), 99.2 (G1), 78.6 (G3), 76.4 (G5), 75.5 (G2), 71.6 (G4), 66.9 (CH₂Ph), 45.5-44.2 (NMe), 25.6-25.5 (Si—C—CH₃), 17.7-13.5-12.6 (Si—C—CH₃) −4.6 −4.8 −4.9 −5.2 (Si—CH₃); m/z (CI) 856 [M+NH₄]⁺.

### Benzyl [4-nitrophenyl-2-[(etoposide-4′-O-carbonyl)methylamino]-2,3,4-tri-O-(tert-butyldimethylsilyl)-β-D-glucopyranosid]uronate 6

DMAP (124.5 mg, 1.035 mmol) was added to a solution of 5 (0.51 g, 0.609 mmol) and etoposide (287 mg, 0.487 mmol) in CH₂Cl₂ (107 mL). Triethylamine (0.14 mL, 1.035 mmol) was added dropwise, and the mixture was stirred 16 h at room temperature. After evaporation, the residue was purified by chromatography (CH₂Cl₂/CH₃CN: 8/2). Prodrug 6 was isolated as a white solid (0.39 g, 57%). C₆₈H₉₄N₂O₂₃Si₃; mp 171° C.; [α]_{D} +1.3 (c 0.99 in CHCl₃); νₘₐₓ/cm⁻¹(CDCl₃) 1770 (CO ester), 1729 (CO carbamate), 1601 (aromatics), 1525, 1348 (NO₂); δ_{H}(CDCl₃) 8.67 (d, Jₘ 2, 1H, a3), 8.10 (dd, J_{O} 9, Jₘ 2, 1H, a5), 7.30 (5H, Ph 7.04 (d, Jₘ 2, 1H, a6), 6.84 (s, 1H, e5), 6.55 (s, 1H, e8), 6.38-6.22 (2H, e2′, e6′), 6.03 (d, J 1, 1H, e10A), 6.02 (s, J 1, 1H, e10B), 5.77 (d, J 6, 1H, G1), 5.16 (AB, J 12, 1H, CH₂(A)Ph), 5.09 (AB, J 12, 1H, CH₂(13)Ph), 4.92 (d, J3, 1H, g4), 4.77 (q, J 5, 1H, g7), 4.68 (d, J 8, 1H, g1), 4.59 (d, J 5.5, 1H, e1), 4.52 (br, 1H, G3), 4.41 (br, 2H, e11A, G4), 4.22-4.15 (2 H, g6equ, e11B), 4.05 (d, J 6, 1H, G2), 3.91 (d, J 3.5, 1H, G5), 3.82-3.68 (7H, g3, OCH₃), 3.59 (t, J 10, 1H, g6ax), 3.44 (t, J 8, 1H, g2), 3.36 (1H, g5), 3.27 (5 H, e2, e4,N—CH₃), 2.86 (m, 1 H, e3), 1.39 (d, J 5, 3H, g8), 0.96 (9 H, Si—C—CH₃), 0.91 (9 H, Si—C—CH₃), 0.89 (9 H, Si—C—CH₃), 0.21-(−0.02) (18. H, Si—CH₃); δ_{C}(CDCl₃) 174.9 (e9), 168.4 (G6), 156.2, 153.5, 153.2, 152.1, 148.9, 147.4, 142.0, 137.6, 135.2, 132.7, 132.5 (C quaternary, carbamate) 128.6, 128.5, 128.3 (Ph), 126.6 (a3), 123.6 (a5), 114.3 (a6), 110.9 (e8), 109.1 (e5), 106.8 (e2′, e6′), 102.1 (e10), 101.7 (g1), 99.9 (g7), 98.1 (G1), 79.8 (g5), 79.0 (G3), 77.0 (G5), 76.8 (G2), 74.6 (g2), 73.9 (g4), 73.1 (g3), 72.4 (G4), 68.1 (g6), 67.9 (e11), 67.0 (CH₂Ph), 66.5 (e4), 56.0 (O—CH3), 44.0 (e1), 41.3 (e2, e3), 37.5, 37.0 (N—CH3), 26.6, 25.9, 25.8, 25.7(Si—C—CH₃), 20.3 (g8), 18.1, 18.0, 17.9 (Si—C—CH_{3,}), −4.2, −4.5, 4.6, 4.7, 4.9, −5.7 (Si—CH₃); m/z (FAB⁺) 1413 [M+Na]⁺.

### Benzyl [4-nitrophenyl-2-[(etoposide-4′-O-carbonyl)ihethylamino]-β-D-glucopyranosid]uronate (7)

To a solution of 6 (223.2 mg, 0.16 mmol) in pyridine (2.65 mL) at 0° C., was added dropwise BH/pyridine (2.65 mL, 70%). The mixture was stirred 4 hours at 0° C., then 10 hours at room temperature. After evaporation, the residue was taken in 200 mL CH₂Cl₂, and washed with water; the aqueous phase is extracted with CH₂Cl₂. The organic phases were dried over magnesium sulfate, and the compound was purified by chromatography (AcCN). Product 7 was obtained as a beige solid (150 mg, 89%). C₅₀H₅₂N₂O₂₃; mp 170° C.; [α]_{D} −9.2 (c 1.1 in CHCl₃); νₘₐₓ/cm⁻¹(CDCl₃) 3406 (O—H), 1752 (CO ester), 1713 (CO carbamate), 1602 (aromatics), 1525, 1346 (NO₂) δ_{H}(CDCl₃) 8.20 (br, 2H, a3, a5), 7.39 (br, 5H, Ph), 7.12 (d, J 9, 1H, a6), 6.95+5.62 (2H, e2′, e6′), 6.65 (s, 1H, e5), 6.57 (s, 1H, e8), 6.07 (s, 2H, e10), 5.34 (AB, J 12, 1H, CH₂(A)Ph), 5.27 (AB, J 12, 1H, CH₂(B)Ph), 5.13 (d, J 6, 1H, G1), 5.03 (d, J 2, 1H, G5), 4.70 (2H, g7, e1), 4.46 (2H, e11), 4.38 (1H, g1), 4.23 (dd, J 10, J 4, 1H, g4), 4.15 (d, J 10, 1H, G4), 3.91 (br, 1H, G3), 3.68 (G2), 3.66-3.52 (4H, e2, e4, g3, g6), 3.51 (s, 9H, OCH₃, NCH₃), 3.42 (1H, g2), 3.33 (1H, g5), 2.99 (1 H, e3), 1.40 (d, J 5, 1H, g8); δ_{C}(CDCl3) 176.4 (e9), 167.5 (G6), 156.0, 152.9, 150.8, 148.0, 146.0, 141.5, 137.4, 134.0, 131.7, 131.3, 126.4, 125.5 (C quaternary, carbamate), 127.8, 127.7, 127.4 (Ph tertiary), 123.3, 122.1 (a3, a5), 113.5 (a6), 110.5 (e8), 109.0 (e5), 107.8 (e2′, e6′), 100.8 (e10, G1), 98.8 (g7), 96.4 (g1), 78.8 (g4), 73.9 (G4, g2), 72.9 (G2), 72.5 (e4), 71.2 (G3), 69.7 (G5), 67.4 (g3, g6), 67.0-66.7 (e11, CH₂Ph), 65.0 (g5), 55.3 (O—CH₃), 43.2 (e1), 338.8-38.1 (e2, e3), 36.6 (N—CH₃), 19.4 (g8) ; m/z (FAB+) 1071 [M+Na]⁺.

### [4-Nitrophenyl2-[(etoposide-4′-O-carbonyl)methylaminol-β-D-glaco-pyranosid]uronic acid (1a)

Palladium-on-charcoal (137 mg, 10%) and 1,4 cyclohexadiene (0.54 mL, 5.7 mmol) were added to an ethanolic solution of 7 (63.6 mg, 0.06 mmol in 1.8 mL). The mixture was stirred at 45° C. for 15 hours. After filtration over celite and evaporation, the crude product was purified by chromatography (CH₃CN/H₂O: 90/10)). Prodrug 1a was isolated as a beige powder (17 mg, 29%). C₄₃H46N₂O₂₃; mp 186° C.; [α]_{D} +6.5 (c 0.85 in MeOH); νₘₐₓ/cm⁻¹(KBr) 3426 (O—H), 1770 (CO ester), 1717 (CO carbamate), 1603 (aromatics), 1505, 1378 (NO₂); δ_{H}(DMSO) 8.40 (1H, a3), 8.17 (d, J 9, 1H, a5), 7.46 (d, J 9, 1H, a6), 7.02 (s, 1H, e5), 6.55 (s, 1H, e8), 6.28 (2H, e2′, e6′), 6.02 (s, 2H, e10), 5.29 (2H, OH), 5.18 (1H, G1), 4.95 (1H, OH), 4.72 (q, J 5, 1H, g7), 4.58 (2H, g1,e1), 4.27 (1H e11A), 4.08 (1H, e11B), 3.66 (s, 6H, OCH₃), 3.62-3.09 (14H, NCH₃, e4, g2, g3, g4,g5, g6, G2, G3, G4, G5), 3.07 (1H, e2), 2.91 (1H, e3), 1.24 (d, J 5, 3H, g8); δ_{C}(DMSO) 175.2 (e9), 172.2 (G6), 158.2, 153.2, 151.8, 148.45, 147.0, 141.6, 139.1, 132.7, 129.6, 128.0 (C quaternary, carbamate), 126.0 (a3), 124.6 (a5), 116.9 (a6), 110.6 (e5), 110.4 (e8), 108.0 (e2′, e6′), 102.2 (g1), 102.0 (e10), 101.9 (G1), 99.9 (g7), 80.8 (g4), 75.0 (e2), 74.4-74.0 (G4, g2), 73.4 (G2), 72.5 (G3), 68.0 (G5) 66.4 (g3, g5, g6, e4), 56.5 (OCH₃), 43.9 (e1), 41.0 (e3), 37.8 (N—CH₃), 21.0 (g8); m/z (ES⁺) 981 [M+Na]⁺, 997 [M+K]⁺.

In Vitro Cytotoxicity

Cytotoxicity was tested against L1210 (mouse leukemic cell line) cells using the MTA assay.

L1210 cells were cultivated in RPMI 1640 medium (Gibco) supplemented with 10% fetal calf serum, 2 mM L-glutaine, 100 units/mL penicillin, 100 g/mL streptomycin, and 10 mM HEPES buffer (pH=7.4). Cytotoxicity was measured by the microculture tetrazolium assay (MTA). Cells were exposed to graded concentrations of drug (nine serial dilutions in triplicate) for 48 h. Results are expressed as IC50, the concentration which reduced by 50% the optical density of treated cells with respect to the optical density of untreated controls.

For the cell cycle analysis, L1210 cells (5×10⁵ cells/mL) were incubated for 21 h with various concentrations of drugs. Cells were then fixed by 70% ethanol (v/v), washed, and incubated in PBS containing 100 μg/mL RNAse and 50 μg/mL propidium iodide for 30 min at 20 C. For each sample, 10 000 cells were analyzed on a XLMCL flow cytometer (Beckman Coulter, France).

HPLC Conditions

Good separation in a short delay was obtained with a reversed-phase Phenyl analytical column (Spherisorb 250×4,6) using isocratic conditions (1 mL/min) of 60% phosphate buffer (0.02 M, pH 3) and 40% acetonitrile with UV detection at 254 nm. Using these conditions the retention time of etoposide, prodrug, and cyclized spacer were 4.9, 3.4, 5.8. min., respectively.

Stability of Compounds in a Buffer Solution

A solution of 500 μl/mL of prodrug 1a in 0.02 M phosphate buffer (pH 7.2) was incubated for various times at 37° C. Aliquots (100 μL) were taken at various times and analyzed by HPLC after dilution with eluent (300 μL).

Enzymatic Cleavage by E. coli β-D-glucuronidase

Hydrolysis was investigated by incubating a solution of 500 μg/mL of prodrug 3 and 20 μg/mL of E. coli β-D-glucuronidase in 0.02 M phosphate buffer (pH 7.2) at 37° C. Aliquots (100 μl) were taken at various times and analyzed by HPLC after dilution with 300 μl of eluent.

1. Compounds having the following formula (I): 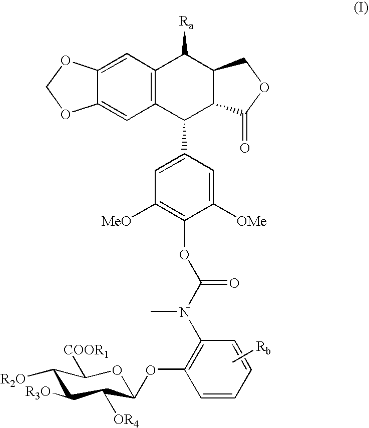
in which:
Rₐ represents a sugar moiety, an arylamino group, or an alkyl group from 1 to 10 carbon atoms comprising at least one amino group,
R_{b} represents an halogen atom, an halogenoalkyl group from 1 to 5 carbon atoms, a nitro group, or a group —NR(COR′) in which R and R′, independently from each other, represent an alkyl group from 1 to 5 carbon atoms,
R₁ represents H, or a protecting group for COOH group,
R₂, R₃, and R₄, independently from each other, represent H, or a protecting group for OH group.

2. Compounds according to claim 1, of formula (I) wherein R₁, R₂, R₃, and R₄ represent H.

3. Compounds according to claim 1, of formula (I) wherein Rₐ represents:
a sugar moiety, selected among the derivatives of glucose, such as the glucose methylacetal of the following: 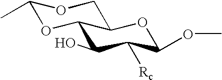
wherein R_{c} represents an hydroxyl or an amino group such as —N(CH₃)₂,
or an arylamino group, and more particularly a group of the following formula:
—HN—C₆H₄R_{d}
wherein R_{d} represents an halogen atom, or a nitro group,
such as the arylamino group selected among 4-nitroaniline, or 4-fluoroaniline,
or an alkyl group from 5 to 10 carbon atoms comprising at least one amino group, and more particularly a linear alkyl chain comprising two nitrogen atoms in the chain, such as the [(dimethylamino)ethyl]N-methylamino)ethyl group.

4. Compounds according to claim 1, of formula (I) wherein R_{b} represents NO₂, F, Cl, CF₃, or a group —NR(COR′) in which R and R′, independently from each other, represent an alkyl group from 1 to 5 carbon atoms.

5. Compounds according to claim 1, having the following formulae: 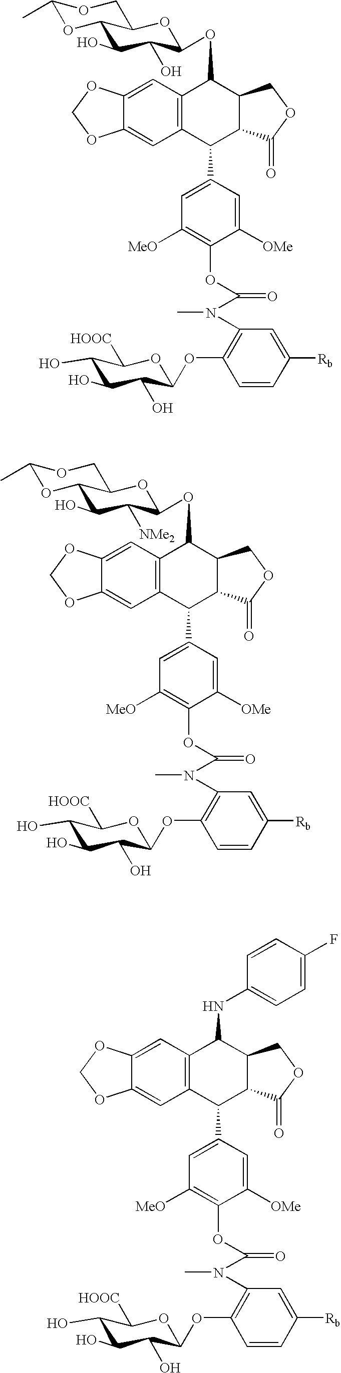 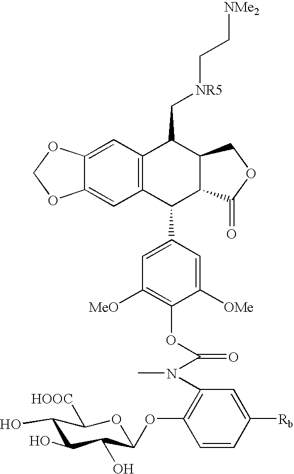
wherein R_{b} represents NO₂, F Cl, CF₃, or a group —NR(COR′) in which R and R′, independently from each other, represents an alkyl group from 1 to 5 carbon atoms, and R₅ represents H or CH₃.

6. Compound according to claim 1, having the following formulae:

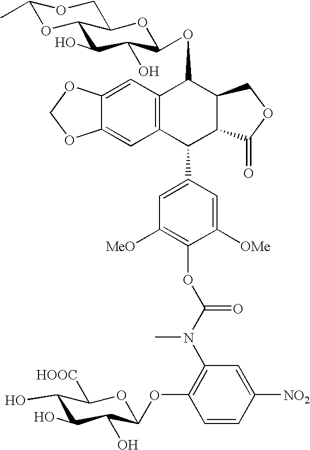

7. Pharmaceutical compositions comprising at least one compound of formula (I) as defined in claim 2, or a salt thereof, in association with a suitable pharmaceutical carrier.

8. Pharmaceutical compositions according to claim 7, comprising at least one of the following compounds: 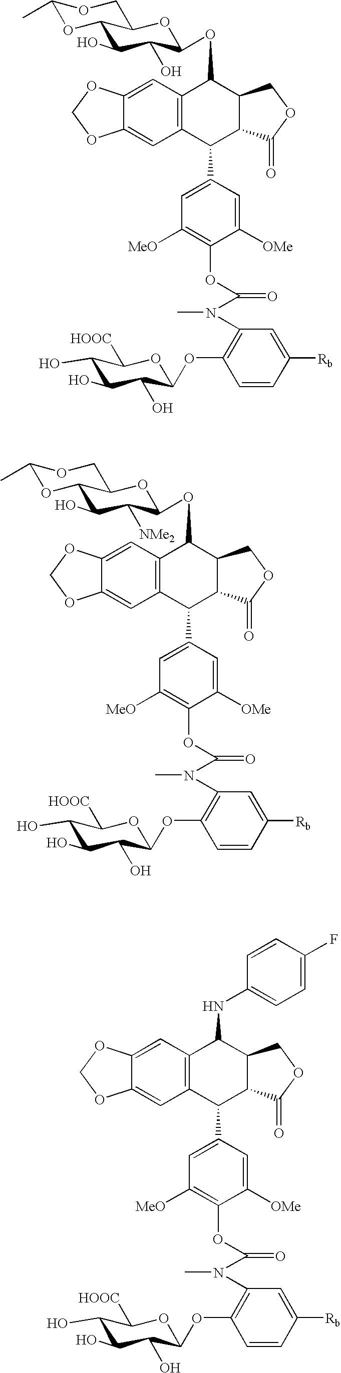 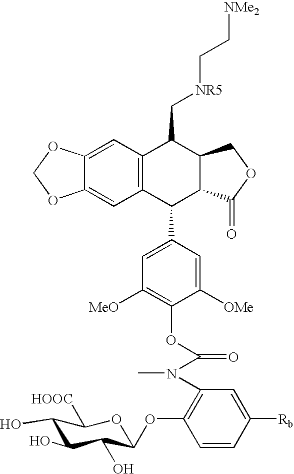
wherein R_{b} represent NO₂, F, Cl, CF₃, or a group —NR(COR′) in which R and R′, independently from each other, represent an alkyl group from 1 to 5 carbon atoms, and R₅ represents H or CH₃.

9. Pharmaceutical composition according to claim 7, comprising at least the following compound:

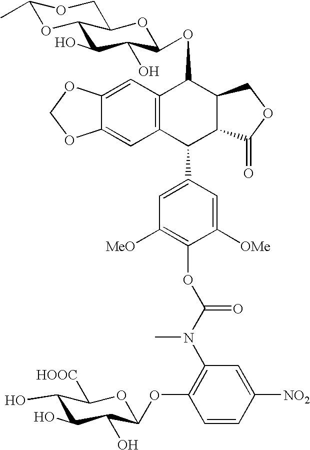

10. Pharmaceutical composition according to claim 7, in a suitable form for oral administration, or for administration by injection, such as intravenous route.

11. Pharmaceutical composition according to claim 7, characterized in that the dosage of the compounds of formula (I) is comprised between approximately 100 mg/m²/day, and approximately 200 mg/m²/day, during approximately 5 days.

12. Use of a compound of formula (I) as defined in claim 2, for the manufacture of a drug for the treatment of cancers such as lung cancer, testicular cancer, Kaposi's sarcoma, lymphoma, and leukemia.

13. Process for the preparation of a compound according to claim 1, characterized in that it comprises the following steps:
amine activation of the following compound of formula A by treatment with phosgene in order to obtain the following compound of formula B: 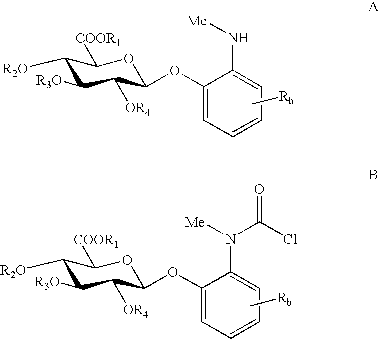
wherein:
R₁ represents a protecting group for COOH group, such as a benzyl or a methyl group,
R₂, R₃, and R₄ represent a protecting group for OH group, such as a terbutyldimethylsilyl or acetate group,
coupling of compound B obtained above, with the following compound of formula C, 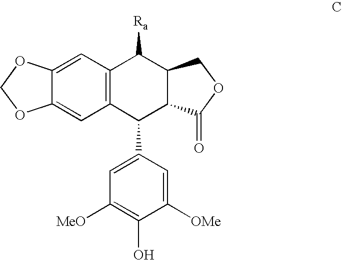
in order to obtain the following compound D: 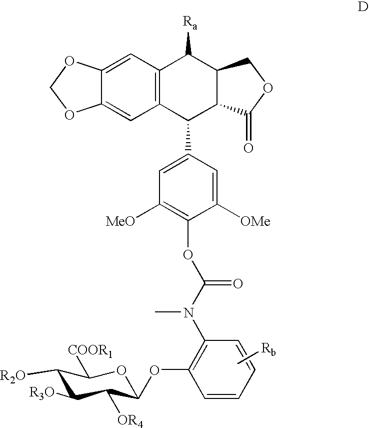
wherein:
R₁ represents a protecting group for COOH group, such as a benzyl or methyl group,
R₂, R₃, and R₄ represent a protecting group for OH group, such as a terbutyldimethylsilyl or acetate group,
deprotection of the OH groups of compound D, for instance with HF/pyridine when R₂, R₃, and R₄ represent a terbutyldimethylsilyl group, in order to obtain the following compound E: 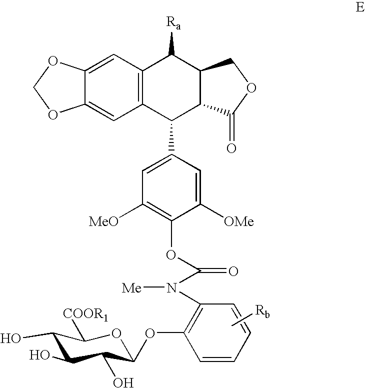
wherein:
R₁ represents a protecting group for COOH group, such as a benzyl or methyl group,
deprotection of the COOH group of compound E, for instance with cyclohexadiene over palladium when R₁ represents a benzyl group, in order to obtain the following compound F:

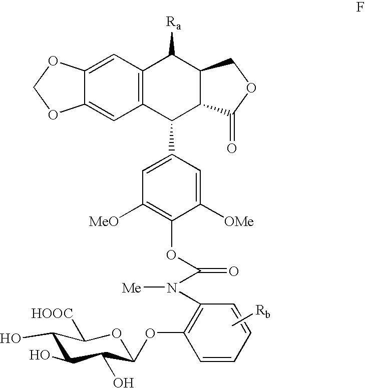

14. Compounds according to claim 1, used as intermediary products said compound corresponding to formula (I) wherein:
R₁ represents a protecting group for COOH group, such as benzyl or methyl group,
and/or, R₂, R₃, and R₄ represent a protecting group for OH group, such as a terbutyldimethylsilyl or acetate group.

15. Compounds according to claim 14, having the following formulae: 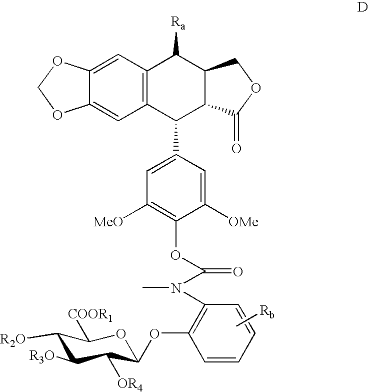
wherein
R₁ represents a protecting group for COOH group, such as benzyl or methyl group,
and R₂, R₃, and R₄ represent a protecting group for OH group, such as a terbutyldimethylsilyl or acetate group, 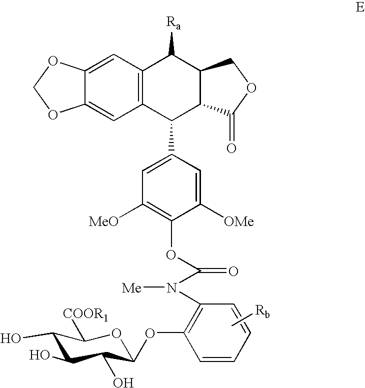
wherein R₁ represents a protecting group for COOH group, such as benzyl or methyl group.
